(19) United States
(12) Patent Application Publication
(10) Pub. No.: US 2025/0194971 A1
(43) Pub. Date: Jun. 19, 2025

(54) Analyte Sensor

(71) Applicant: PercuSense, Inc., Valencia, CA (US)

(72) Inventors: Kaitlynn Olczak, Santa Clarita, CA (US); Blake Morrow, Los Angeles, CA (US); Rajiv Shah, Rancho Palos Verdes, CA (US); Brian Kannard, Los Angeles, CA (US); Bradley Liang, Bloomfield Hills, MI (US); Katherine Wolfe, Dunwoody, GA (US); Elizabeth Espinoza, Northridge, CA (US)

(73) Assignee: PercuSense, Inc., Valencia, CA (US)

(21) Appl. No.: 18/988,687

(22) Filed: Dec. 19, 2024

Related U.S. Application Data
(60) Provisional application No. 63/612,015, filed on Dec. 19, 2023.

(51) Int. Cl. A61B 5/1486; A61B 5/145; C12Q 1/00

(52) U.S. Cl. CPC A61B 5/14865; A61B 5/14532; C12Q 1/003; C12Q 1/006

(57) ABSTRACT

In one embodiment, a working electrode to detect the presence of an analyte is disclosed. The working electrode includes an electrical insulator having a well. The working further includes an electrical conductor that is positioned at the bottom of the well and a reactive chemistry that includes a dehydrogenase based enzyme and cofactor at least partially filling the well. Where the reactive chemistry has a minimum molecular weight and a hydrogel is located over the reactive chemistry. The hydrogel is in contact with the insulation and the reactive chemistry and the hydrogel has a porosity less than the minimum molecular weight of the reactive chemistry.

### RELATED APPLICATIONS

This application claims the benefit of U.S. provisional application No. 63/612,015, filed Dec. 19, 2023. The application listed above is hereby incorporated by reference in its entirety for all purposes.

### FIELD OF THE INVENTION

The invention generally relates to systems and methods for real time monitoring of physiological parameters to enable monitoring of physical conditions.

### BACKGROUND OF THE INVENTION

Awareness of the importance of metabolic health is growing as its association with chronic conditions such as type 2 diabetes and heart disease becomes better understood. Continuous glucose monitoring (CGM) can provide some insight regarding metabolic health, monitoring one or more additional metabolic analytes such as lactate, oxygen or ketones can provide additional insight.

Accordingly, it may be highly desirable to develop sensors capable of detecting a variety of analytes, particularly those associated with metabolic health. Moreover, because of the variety of enzymes that have been developed, it may be highly desirable to have flexibility regarding the types of enzymes that can be used on a sensor. In many examples discussed below an electrode configuration is disclosed that may be used. Additionally, the various electrode configurations can be used to detect a single or multiple analytes, including those associated with metabolic health and/or diabetes. While embodiments and examples may be related to particular analytes and electrode configurations, the scope of the disclosure and claims should not be construed to be limited to the specifically addressed analyte, enzymes and electrode configurations discussed below. Rather it should be recognized that additional or other analytes, enzymes or electrode configurations can be used and considered within the scope of this disclosure.

### BRIEF SUMMARY OF THE INVENTION

In one embodiment, a working electrode to detect the presence of an analyte is disclosed. The working electrode includes an electrical insulator having a well. The working further includes an electrical conductor that is positioned at the bottom of the well and a reactive chemistry that includes a dehydrogenase based enzyme and cofactor at least partially filling the well. Where the reactive chemistry has a minimum molecular weight and a hydrogel is located over the reactive chemistry. The hydrogel is in contact with the insulation and the reactive chemistry and the hydrogel has a porosity less than the minimum molecular weight of the reactive chemistry.

In another embodiment, an electrochemical sensor to detect the presence of an analyte is disclosed. The electrochemical sensor includes a working electrode that has an electrical insulator having a working electrode well and a first electrical conductor being positioned at a bottom of the working electrode well. The electrochemical sensor further includes a reactive chemistry that includes a dehydrogenase based enzyme and cofactor at least partially filling the working electrode well. The reactive chemistry has a minimum molecular weight and a hydrogel is positioned over the reactive chemistry. Additionally, the hydrogel is in contact with the insulation and the reactive chemistry, and further has a porosity less than the minimum molecular weight of the reactive chemistry. The electrochemical sensor further includes a combined counter/reference electrode that includes the electrical insulator that has a counter/reference well and a second electrical conductor being positioned at a bottom of the counter/reference well.

Other features and advantages of the invention will become apparent from the following detailed description, taken in conjunction with the accompanying drawings that illustrate, by way of example, various features of embodiments of the invention.

Methods and structures disclosed herein for treating a user/patient also cover analogous methods and structures performed on, or placed on, a simulated patient, which can be useful, for example, for training, demonstration, procedure and/or device development, and the like. For example, a simulated patient can be physical, virtual, or a combination of physical and virtual. A simulation can include a simulation of all or a portion of a patient, such as an entire body, a portion of a body, a system, an organ, or any combination thereof. Physical elements can be natural, including human or animal cadavers, or portions thereof; synthetic; or any combination of natural and synthetic. Virtual elements can be entirely in silicon, or overlaid on one or more of the physical components. Virtual elements can be presented on any combination of screens, headsets, holographically, projected, loudspeakers, headphones, pressure transducers, temperature transducers, or using any combination of suitable technologies.

### BRIEF DESCRIPTION OF THE DRAWINGS

FIGS. 1A and 2A are exemplary illustrations of a top view of a portion of a sensor, in accordance with embodiments of the present invention.

FIGS. 1B and 2B are exemplary cross-section views of the sensor 100 along section lines A-A in FIGS. 1A and 2A, respectively, in accordance with embodiments of the present invention.

FIGS. 3A-3D are exemplary block diagrams illustrating various electrode configurations of analyte sensor probes, in accordance with various embodiments of the present invention.

FIGS. 4A-4C are different embodiments of working electrodes made in accordance with embodiments of the present invention.

FIG. 5 is a flow chart illustrating exemplary operations to form a working electrode in accordance with embodiments of the present invention.

### DETAILED DESCRIPTION

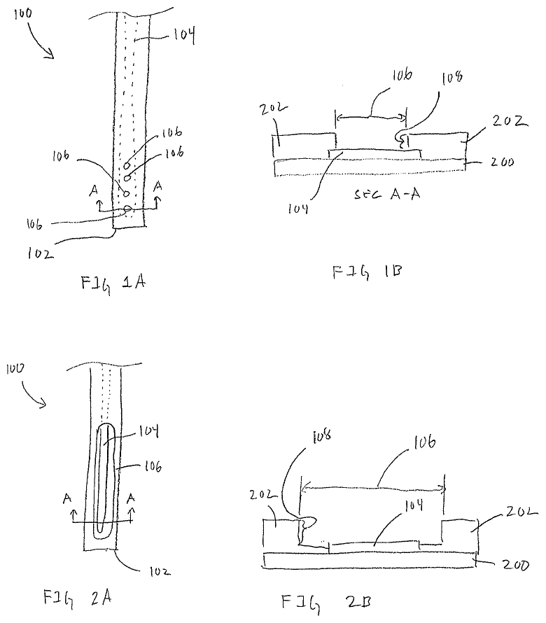

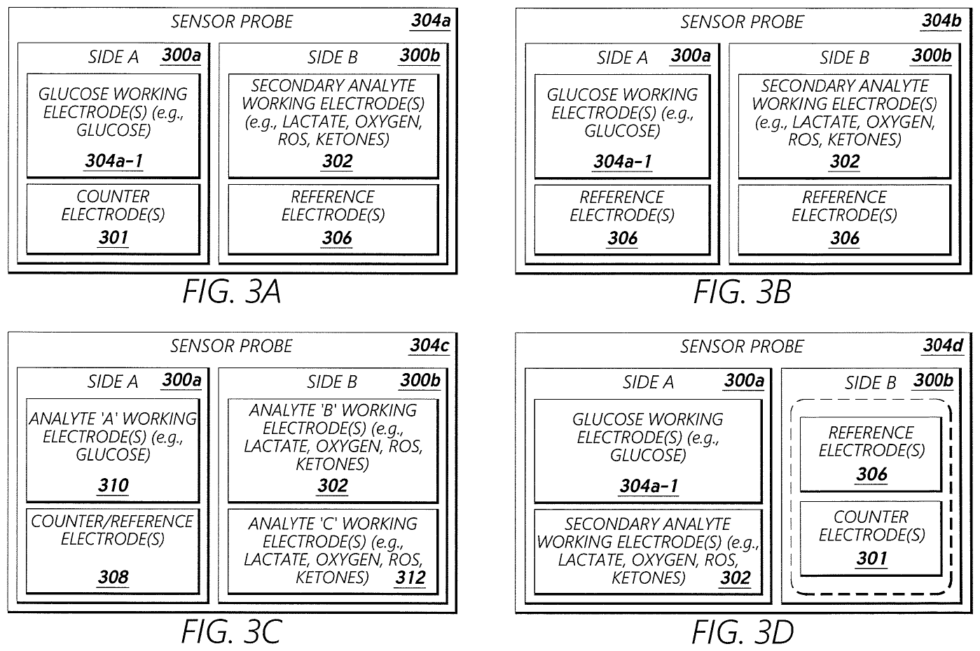

FIGS. 1A and 2A are exemplary illustrations of a top view of a portion of a sensor probe 100, in accordance with embodiments of the present invention. The portion of the sensor probe 100 illustrated in FIGS. 1A and 2A is a distal end 102 of the sensor probe 100, or simply probe 100. The distal end 102 is configured to be placed within a subject while a proximal end (not shown) interfaces with electronic components and remains outside of the subject. In preferred embodiments, the distal end 102 is inserted through the skin and placed within subcutaneous tissue of a subject. However, in other embodiments, the sensor probe 100 may be placed in alternative locations such as, but not limited to the vasculature, organs or other tissue within the subject.

Sensor probe 100 includes a conductor 104 that is disposed or placed between layers of electrically non-conductive insulating materials. In many embodiments, one or more openings 106 (alternatively referred to as apertures throughout this paper) are formed in at least one layer of the non-conductive insulating material to expose the underlying conductor 104. For simplicity FIGS. 1A and 2A include an illustration of the conductor 104 that in many embodiments may be used or configured as a working electrode. Additional conductors may be located within the sensor probe 100 to operate as one or more additional working electrodes, or to be used as a counter electrode, a reference electrode, or combined counter/reference electrode.

FIGS. 1B and 2B are exemplary cross-section views of the sensor probe 100 along section lines A-A in FIGS. 1A and 2A, respectively, in accordance with embodiments of the present invention. In FIGS. 1B and 2B, the conductor 104 is shown sandwiched or located between insulation 200 and insulation 202. Comparing FIGS. 1B and 2B, the aperture 106 is shown having varying width. In various embodiments, the aperture 106 may be less than the width of the conductor 104. In other embodiments, the aperture 106 may be wider than the width of the conductor 104. In still other embodiments, the aperture 106 may be substantially equal to the width of the conductor 104.

As illustrated in FIGS. 1B and 2B, a well depth 108 is established between a top 202a of the insulation 202 and a lower point 202b near the conductor 104. In some embodiments, the well depth 108 may extend below a top 104a of the conductor 104. In these embodiments, insulation 200 and 202 may be removed to expose one or more sides 104b of the conductor 104. The electrode configurations illustrated in FIGS. 1A through 2B are intended to be illustrative of embodiments that enable selective exposure of an electrical conductor. The exposure of the electrical conductor enables the formation of an electrode on the electrical conductor such as a working electrode, a counter electrode, a reference electrode or a combined counter/reference electrode.

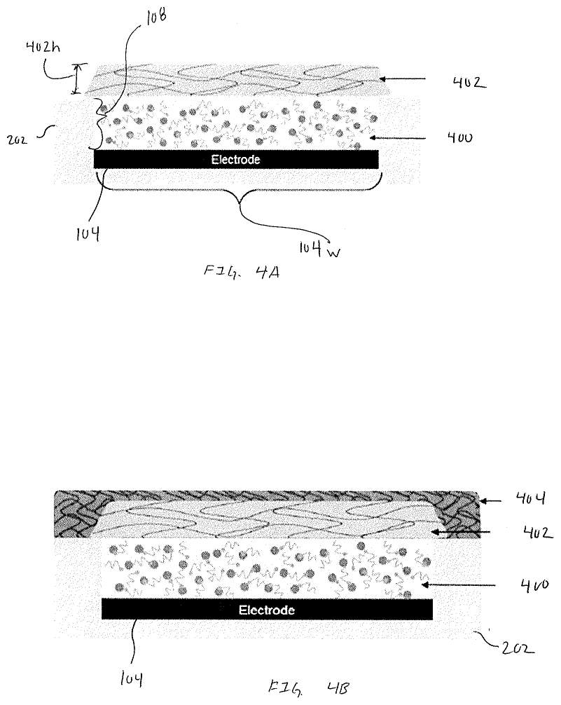

FIGS. 3A-3D are exemplary block diagrams illustrating various electrode configurations of the sensor probe 100, in accordance with various embodiments of the present invention. Exemplary sensor probes 100 of the present disclosure may have configurations of any of the analyte sensors illustrated in FIGS. 304A-304D or various combinations thereof. The sensor probes 304a-304d illustrated in FIGS. 3A-3D each include two sides/faces, namely an ‘A-side’ 300a and a ‘B-side’ 300b. For example, the sensor probes 304a-304d can have a thin, flat, elongated body with a generally rectangular cross-section, wherein the sensor probes 304a-304d have two relatively broad, flat faces on opposite sides, which are parallel and define the primary surface area of the probe. Edges may generally run along the length of the probes 304a-304d where the two faces meet, forming two elongated, relatively narrow surfaces. Sides ‘A’ and ‘B’ of the sensor probes 304a-304d may correspond to the opposite-facing primary surfaces/faces of the respective probes.

Each side of the sensor probes 304a-304d may be configured with one or more electrodes, wherein each electrode can be configured as one of a working electrode, a counter electrode, a reference electrode, or a combined counter and reference electrode. In some embodiments, a two-electrode configuration is implemented, wherein a working electrode is operated with a combined counter and reference electrode. In other embodiments, a three-electrode configuration may be implemented, wherein a working electrode is operated with a separate counter electrode and a separate reference electrode. In some embodiments of a two-electrode system, multiple working electrodes operated with the same polarity may share a combined counter and reference electrode. Similarly, in some embodiments of a three-electrode system, multiple working electrodes operated with the same polarity may share a counter electrode and a reference electrode. In some embodiments of a three-electrode system, multiple electrodes operated with different polarities may have separate counter electrodes and share a reference electrode.

FIG. 3A is an exemplary illustration of a three-electrode sensor probe configuration 304a, wherein the A-side 300a includes a glucose working electrode 304a-1 and one or more counter electrodes 301. The B-side 300b is configured with a second working electrode 302 configured to measure or detect a second analyte and further includes one or more reference electrodes 306.

FIG. 3B is an exemplary illustration of a three-electrode sensor probe configuration 304b, wherein the A-side 300a includes a glucose working electrode 304a-1 and one or more reference electrodes 306. The B-side 300b is configured to measure or detect a second analyte 302 and further includes one or more counter electrodes 304.

FIG. 3C is an exemplary illustration of a two-electrode sensor probe configuration 304c, wherein the A-side 300a is configured with a first working electrode to measure a first analyte 310 and further includes a combined counter and reference electrode. The B-side 300b is configured with a second working electrode 302 and a third working electrode 312. In various embodiments, the second working electrode 302 may be configured to detect the same or a different analyte than the first working electrode. Similarly, the third working electrode 312 may be configured to detect the same or a different analyte than either the first working electrode 310 and/or the second working electrode 302.

FIG. 3D is an exemplary illustration of a three-electrode sensor probe configuration 304d, wherein the A-side 300a includes a glucose working electrode 104a-1 and a second working electrode 302. The B-side 300b is configured with one or more reference electrodes 306 and one or more counter electrodes 304. The permutations illustrated in FIGS. 3A-3D and described above should be construed as exemplary rather than limiting. For example, in some embodiments a two-electrode system may be implemented on the same analyte sensor 104 as a three-electrode system. In such an embodiment the first and second working electrodes may be formed on the A-side while the B-side includes a combined counter and reference electrode along with a discrete counter electrode and a discrete reference electrode.

Any of the example configurations of FIGS. 3A-3D may be implemented in connection with embodiments of the present disclosure. In a given implementation, the positions of the electrodes may advantageously be optimized for accurate single or multi-analyte sensing by leveraging the geometry of the probe and the electrochemical requirements of each electrode. For example, the glucose electrode may desirably be positioned near the tip on the A-side, ensuring direct exposure to interstitial fluid in highly vascularized regions for consistent glucose measurements. The lactate electrode may also be positioned on the A-side, located slightly proximal to the glucose electrode, allowing it to measure lactate levels reflective of metabolic activity downstream from glucose uptake. An oxygen electrode, where present, may be positioned near the middle of the B-side of the probe, separated from the glucose and lactate electrodes to avoid interference from their electrochemical reactions while providing critical oxygen measurements that contextualize metabolic activity. The reference electrode may be placed centrally along the probe's B-side, such as equidistant from the working electrodes on both sides, to maintain a stable potential across all measurements. The counter electrode, where separate from the reference electrode, may be positioned at the proximal end of the B-side, providing uniform current flow for all working electrodes while minimizing interference with analyte detection. This example beneficial arrangements, and/or aspects or considerations associated therewith, can advantageously provide reduced cross-interference, stable signal acquisition, and/or efficient use of the probe's surface area, enabling robust, synchronized multi-analyte sensing with a single implantable device.

The sensor configurations disclosed above and throughout the remainder of this disclosure may be implemented as a working electrode on various combinations of the sensor probes discussed above. In many embodiments discussed below, the working electrode is formed by applying a mobile enzyme in close proximity to the conductor 104. In many embodiments, the mobile enzyme is applied directly to the conductor 104. In other embodiments, the conductor 104 is prepared with a surface treatment or multiple surface treatments. Exemplary surface treatments that may be applied to the conductor include, but are not limited to application of adhesives, anodizing, plasma electrolytic oxidation, plating, electrophoretic deposition and electrochemical assisted methods and techniques. In preferred embodiments, the surface treatment is a plating operation that enhances or improves the surface area of the electrode surface. An exemplary, non-limiting surface treatment capable of increasing the surface area of the electrode surface is electroplating platinum black onto the conductor.

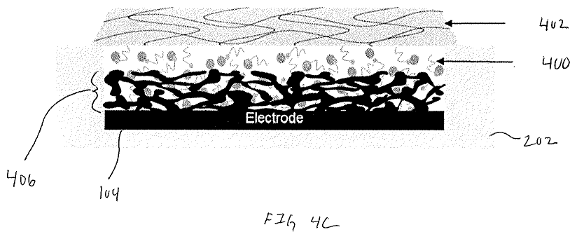

FIGS. 4A-4C are different embodiments of working electrodes made in accordance with embodiments of the present invention. In preferred embodiments, the inclusion of a reactive chemistry 400 enables the electrodes discussed in FIGS. 1A and 2A to be utilized as working electrodes. In preferred embodiments, the reactive chemistry 400 includes a mobile enzyme. For example, in many embodiments, the mobile enzyme is a dehydrogenase enzyme, such as, but not limited to glucose dehydrogenase or 3-hydroxybutyrate dehydrogenase (3HBDH). In embodiments where the mobile enzyme is a dehydrogenase based enzyme it may be advantageous to include a cofactor, cofactor analog, or cofactor derivative. Exemplary, non-limiting cofactors that may be included with a dehydrogenase enzyme are nicotinamide adenine dinucleotide (NAD), nicotinamide adenine dinucleotide phosphate (NADP) or flavin adenine dinucleotide (FAD). In various embodiments, it may be advantageous to modify any cofactor by attaching it to another molecule or polymer to adjust or tune physical characteristics such as, but not limited to molecular weight. The tuning of the combined enzyme and attached cofactor for a specific molecular weight or size can assist in enabling entrapment of the combined enzyme and cofactor.

In many embodiments, the reactive chemistry 400 is retained by the conductor 104 and the insulation 102. Moreover, the reactive chemistry 400 at least partially fills the well depth 108 that is defined between the conductor 104 and the top of the insulation 202. As illustrated in FIG. 4A, the reactive chemistry 400 substantially fills the well depth 108. Being partially retained within the well by the conductor 104 places the reactive chemistry 400 in direct contact with the conductor 104. In many embodiments a hydrogel 402 is applied directly over and completely covering the reactive chemistry 400. Note that as illustrated, the hydrogel 402 is in direct contact with both the insulation 202 and the reactive chemistry 400. Accordingly, the hydrogel 402, along with the insulation 202, and conductor 104 contains or retains the reactive chemistry 400 within the well.

In preferred embodiments, selection or design of the hydrogel 402 is at least partially based on the molecular size or weight of the reactive chemistry 400. For example, in many embodiments the hydrogel is selected to have a molecular weight between 20 k and 2000 k. In other embodiments, the hydrogel is selected to have a molecular weight between 250 k and 1500 k. Recall that the reactive chemistry 400 includes both the mobile enzyme and cofactor and that the reactive chemistry can remain mobile, albeit optionally tuned to a particular molecular weight that will be entrapped or ensnared or partially immobilized by the hydrogel 402. In other embodiments the selection of the hydrogel 402 is at least partially based on the properties of a preferred molecule of interest. For example, the hydrogel 402 may be selected to have physical properties (e.g. hydrophobicity, charge, etc.) to increase to decrease the transport of the reactant/analyte or byproduct of a reaction.

Though not illustrated, in some embodiments, the reactive chemistry 400 may not entirely fill the well depth 108. However, the hydrogel 402 would remain in contact with both the insulation 202 and the reactive chemistry 400. Accordingly, in embodiments where the reactive chemistry 400 does not completely fill the well, the hydrogel 402 may partially fill the well depth. The hydrogel 402 retains or contains the reactive chemistry 400 within the well while also enabling the combined mobile enzyme and cofactor to remain mobile while in close proximity to the conductor 104. Additionally, the hydrogel 402 enables reactants from fluid surrounding the working electrode to be transported through the hydrogel 402 in order to interact with the reactive chemistry 400. In many preferred embodiments, the hydrogel 402 is a highly porous hydrogel selected from the polyhydroxethylmethacrylate (pHEMA) family.

The conductor 104 has an electrode width 104w that can be between 0.005 inches and 0.025 inches. In preferred embodiments, the electrode width 104w is between 0.008 inches and 0.020 inches. Additionally, the well depth 108 can be between 0.0001 inches and 0.010 inches, with preferred embodiments the well depth 108 is between 0.0005 inches and 0.005 inches. Moreover, the hydrogel 402 has a hydrogel height 402h between 0.0005 inches and 0.05 inches with a preferred range between 0.001 inches and 0.01 inches.

FIG. 4B is an exemplary illustration of an alternate embodiment of a working electrode, in accordance with embodiments of the present invention. The working electrode in FIG. 4B includes the insulation 202 and the conductor 104. Moreover, the reactive chemistry 400 is retained within the well by the insulation 202, the conductor 104 and the hydrogel 402. Additionally, the working electrode in FIG. 4B includes a crosslinked hydrogel 404. The crosslinked hydrogel 404 is directly in contact with the insulation 202 and the hydrogel 402. The crosslinked hydrogel 404 may be applied across the entire electrode surface, as illustrated. In other embodiments, the crosslinked hydrogel 404 may not extend to the edges of the sensor. Irrespective of the coverage toward the lateral edges of the sensor, the crosslinked hydrogel can work in conjunction with the hydrogel 402 to retain, contain or entrap the reactive chemistry 400. The inclusion of the crosslinked hydrogel 404 over the hydrogel 402 provides an additional level of containment for the reactive chemistry 400.

In other embodiments, the crosslinked hydrogel 404 may be selected based on factors that enable modification or tuning of flux or transport of a particular molecule of interest (e.g., the analyte of interest, a reactant, or byproduct of a reaction). In some of these embodiments, the crosslinked hydrogel 404 is only applied over the hydrogel 402 and is not in contact with the insulation 202. In still other embodiments, the crosslinked hydrogel 404 may be selected based on adhesion properties to the hydrogel 402. In alternate embodiments, additional adhesion layers that are not illustrated may be optionally used between the layers discussed above.

FIG. 4C is an exemplary illustration of an alternate embodiment of a working electrode, in accordance with embodiments of the present invention. FIG. 4C includes a porous electrode surface 406 that is formed on the conductor 104. In some embodiments the porous electrode surface 406 is generated using an electrochemical process such as electroplating. The porous electrode surface 406 increases the surface area that is available to interact with reactants that are transported through the hydrogel 402 to the reactive chemistry 400.

Accordingly, while the description above refers to particular embodiments of the invention, it will be understood that many modifications may be made without departing from the spirit thereof. In particular, while many embodiments are directed toward specific combinations of analytes and physical sensor data, it should be understood that where possible, each embodiment is capable of being combined with each and every other embodiment. The presently disclosed embodiments are therefore to be considered in all respects as illustrative and not restrictive, the scope of the invention being indicated by the appended claims, rather than the foregoing description, and all changes that come within the meaning and range of equivalency of the claims are therefore intended to be embraced therein.

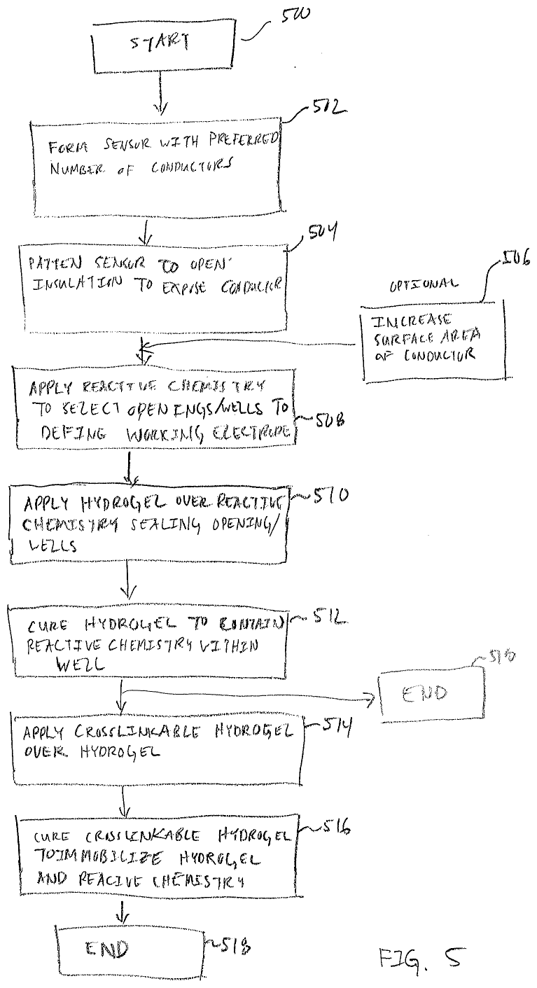

FIG. 5 is a flow chart illustrating exemplary operations to form a working electrode in accordance with embodiments of the present invention. The flow chart begins with a start operation 500. Operation 502 forms a sensor with the desired number of conductors. In many embodiments for the electrochemical detection of analytes the number of conductors depends on the number of analytes being detected, whether a two cell (i.e., working electrode and shared counter and reference electrode) or three cell (i.e., working electrode, reference electrode and counter electrode) system is being used, and if multiple analytes are being detected, if any of the working electrodes share a counter electrode, reference electrode or combined counter/reference electrode. Operation 504 patterns the sensor to create openings or well in insulation that expose portions of the conductors that will be formed into a working electrode, reference electrode, counter electrode or combined counter/reference electrode. Optional operation 506 increases the surface area of the conductor exposed via operation 504.

Operation 508 applies reactive chemistry within select openings or wells to define the working electrode(s). In many of these embodiments the reactive chemistry includes a mobile enzyme from the dehydrogenase family and a cofactor. Operation 510 applies a hydrogel over the reactive chemistry and operation 512 cures the hydrogel thereby sealing the reactive chemistry within the opening or well. In some embodiments, after operation 512, end operation 518 completes the flow chart. In other embodiments, optional operation 514 applies a crosslinkable hydrogel over the hydrogel and optional operation 516 cures the crosslinkable hydrogel immobilizing the hydrogel and the reactive chemistry. End operation 518 completes the flow chart.

Although certain preferred examples are disclosed above, it should be understood that the inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, various examples may be carried out in a manner that achieves or optimizes one advantage or group of advantages as taught herein without necessarily achieving other aspects or advantages as may also be taught or suggested herein.

Certain spatially relative terms, such as “outer,” “inner,” “upper,” “lower,” “below,” “above,” “vertical,” “horizontal,” “top,” “bottom,” “distal,” “proximal,” and similar terms, are used herein to describe a spatial relationship of one device/element or anatomical structure to another device/element or anatomical structure. It should be understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa. It should be understood that spatially relative terms, including those listed above, may be understood relative to a respective illustrated orientation of a referenced figure.

Certain reference numbers are re-used across different figures of the figure set of the present disclosure as a matter of convenience for devices, components, systems, features, and/or modules having features that are similar in one or more respects. However, with respect to any of the examples disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

1. An electrochemical sensor to detect the presence of an analyte, comprising:
a working electrode that includes,
an electrical insulator having a working electrode well;
an first electrical conductor being positioned at a bottom of the working electrode well;
a reactive chemistry that includes a dehydrogenase based enzyme and cofactor at least partially filling the working electrode well, the reactive chemistry having a minimum molecular weight;
a hydrogel being in contact with the insulation and the reactive chemistry, the hydrogel having a porosity less than the minimum molecular weight of the reactive chemistry, and
a combined counter/reference electrode that includes,
the electrical insulator having a counter/reference well;
a second electrical conductor being positioned at a bottom of the counter/reference well.

2. The electrochemical sensor of claim 1, further comprising:
a crosslinkable hydrogel that covers the hydrogel, the crosslinkable hydrogel being in contact with the insulation and the hydrogel and further covering the reactive chemistry and conductor.

3. The electrochemical sensor of claim 2, wherein the working electrode is formed on an A-side and the combined counter/reference electrode is formed on a B-side.

4. The electrochemical sensor of claim 2, wherein the working electrode is formed on an A-side and the combined counter/reference electrode is formed on the A-side.

5. A working electrode to detect the presence of an analyte, comprising:
an electrical insulator having a well;
an electrical conductor being positioned at a bottom of the well;
a reactive chemistry that includes a dehydrogenase based enzyme and cofactor at least partially filling the well, the reactive chemistry having a minimum molecular weight, and
a hydrogel being placed over the reactive chemistry, the hydrogel being in contact with the insulation and the reactive chemistry and having a porosity less than the minimum molecular weight of the reactive chemistry.

6. The working electrode of claim 5, further comprising:
a crosslinkable hydrogel that covers the hydrogel, the crosslinkable hydrogel being in contact with the insulation and the hydrogel and further covering the reactive chemistry and conductor.

7. The working electrode of claim 6, wherein the crosslinkable hydrogel extends to lateral edges of the insulation.

8. The working electrode of claim 6, wherein the crosslinkable hydrogel does not extend to lateral edges of the insulation.

9. The working electrode of claim 6, wherein the hydrogel is selected from the pHEMA family.

10. The working electrode of claim 9, wherein the cofactor is NAD or NADP.
